(19) United States
(12) Patent Application Publication
(10) Pub. No.: US 2022/0277241 A1
(43) Pub. Date: Sep. 1, 2022

(54) METHOD AND SYSTEM FOR ASSIGNING RESOURCES IN PRODUCT DEVELOPMENT

(71) Applicant: AIZEN ALGO PRIVATE LIMITED, Telangana (IN)

(72) Inventors: Usha Balakrishnan BITHUNSHAL, Telangana (IN); Ramdas MANAKKOTE, Telangana (IN); Srinivas Varma MUDUNURI, Telangana (IN); Varma Srinivasa Rajasekara RUDRARAJU, Telangana (IN)

(21) Appl. No.: 17/636,503

(22) PCT Filed: Aug. 18, 2020
(86) PCT No.: PCT/IN2020/050721

§ 371 Date: Feb. 18, 2022

(30) Foreign Application Priority Data
Aug. 18, 2019 (IN) 201941006375

(51) Int. Cl. G06Q 10/06; G16H 20/10; G16H 40/20

(52) U.S. Cl. CPC G06Q 10/06313; G16H 20/10; G16H 40/20

(57) ABSTRACT

The present disclosure relate to a method and system to assign resources for development of a pharmaceutical product. The method comprises receiving one or more inputs associated with the pharmaceutical product from a user and determining a complexity score of the pharmaceutical product based on the one or more inputs received. The method further comprises classifying the pharmaceutical product based on the complexity score and determining a timeline for development of the pharmaceutical product based on the classification. The method also comprises assigning the resources for development of the pharmaceutical product based on the determination, wherein the resources include at least one of project members and equipment's required for development of the pharmaceutical product.

### CROSS REFERENCE TO RELATED APPLICATION

This application claims the benefit of priority to Indian Provisional Patent Application Number 201941006375, filed on 18 Aug. 2019, the entire contents of which are hereby incorporated by reference.

### TECHNICAL FIELD

Embodiments of the present disclosure are related, in general to project management, and more particularly, but not exclusively to a method and system for assigning resources for a product development based on complexity of the product.

### BACKGROUND

Currently, in any research and development (R&D) industry, project management is an important activity and one of the challenges in the project management or project planning is the optimum or effective assignment and utilization of available resources. In the R&D industry, an approximate timeline and resources are required for completion of a product development to effectively assign resources for the project. Predicting the approximate timeline for the product development is a difficult task as complexity involved in the product development is unpredictable by normal human knowledge and experience. Complexity evaluation of the product to be developed is currently carried out by individuals and is prone to the human error in a great extent. A minor mistake in the complexity evaluation will lead to mistakes in timeline preparation and resource allocation, which thereby leads to loss and effect on delivery of the product. Consideration of all properties of the product may help in efficient evaluation of complexity for the product development. The existing complexity evaluation methods do not use all the properties of the product to be developed for evaluating the complexity of the product development. The effective assignment of resources and timeline estimation of product completion is possible only with accurate determination of complexity. Currently, there is no such system and technique available that accurately determines the complexity of product to be developed and assigning resources based on the complexity of the product.

Accordingly, there is required an intelligent technique to provide a method and system for assigning resources for the product development.

The information disclosed in this background of the disclosure section is only for enhancement of understanding of the general background of the disclosure and should not be taken as an acknowledgment or any form of suggestion that this information forms prior art already known to a person skilled in the art.

### SUMMARY

Embodiments of the present disclosure relate to a method of assigning resources for development of a pharmaceutical product. The method comprises receiving one or more inputs associated with the pharmaceutical product from a user and determining a complexity score of the pharmaceutical product based on the one or more inputs received. The method further comprises classifying the pharmaceutical product based on the complexity score and determining a timeline for development of the pharmaceutical product based on the classification. The method also comprises assigning the resources for development of the pharmaceutical product based on the determination, wherein the resources include at least one of project members and equipment's required for development of the pharmaceutical product.

Another aspect of the present disclosure relates to a system to assign resources for development of a pharmaceutical product. The system comprises a memory and a processor. The processor is configured to receive one or more inputs associated with the pharmaceutical product from a user, and determine a complexity score of the pharmaceutical product based on one or more inputs received. The processor is further configured to classify the pharmaceutical product based on the complexity score, and determine a timeline for development of the pharmaceutical product based on the classification. The processor is also configured to assign the resources for development of the pharmaceutical product based on the determination, wherein the resources include at least one of project members and equipment's required for development of the pharmaceutical product.

The system, and associated method of the present disclosure overcome one or more of the shortcomings of the prior art. Additional features and advantages may be realized through the techniques of the present disclosure. Other embodiments and aspects of the disclosure are described in detail herein and are considered a part of the claimed disclosure.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF DRAWINGS

The accompanying drawings, which are incorporated in and constitute a part of this disclosure, illustrate exemplary embodiments and, together with the description, serve to explain the disclosed principles. In the figures, the left-most digit(s) of a reference number identifies the figure in which the reference number first appears. The same numbers are used throughout the figures to reference like features and components. Some embodiments of device or system and/or methods in accordance with embodiments of the present subject matter are now described, by way of example only, and with reference to the accompanying figures, in which:

FIG. 1 illustrates an exemplary architecture of a proposed system, to assign resources for product development in accordance with some embodiments of the present disclosure;

FIG. 2 illustrates a block diagram of project management system of FIG. 1 in accordance with some embodiments of the present disclosure;

FIG. 3A illustrates an exemplary flow diagram to assign resources for product development in accordance with an embodiment of the present disclosure;

FIG. 3B illustrates an exemplary flow diagram to evaluate complexity of product in accordance with an embodiment of the present disclosure; and

FIG. 4 illustrates a block diagram of an exemplary computer system for implementing embodiments consistent with the present disclosure.

The figures depict embodiments of the disclosure for purposes of illustration only. One skilled in the art will readily recognize from the following description that alternative embodiments of the structures and methods illustrated herein may be employed without departing from the principles of the disclosure described herein.

### DETAILED DESCRIPTION

In the present document, the word “exemplary” is used herein to mean “serving as an example, instance, or illustration.” Any embodiment or implementation of the present subject matter described herein as “exemplary” is not necessarily to be construed as preferred or advantageous over other embodiments.

While the disclosure is susceptible to various modifications and alternative forms, specific embodiment thereof has been shown by way of example in the drawings and will be described in detail below. It should be understood, however that it is not intended to limit the disclosure to the particular forms disclosed, but on the contrary, the disclosure is to cover all modifications, equivalents, and alternative falling within the spirit and the scope of the disclosure.

The terms “comprises”, “comprising”, or any other variations thereof, are intended to cover a non-exclusive inclusion, such that a setup, device or method that comprises a list of components or steps does not include only those components or steps but may include other components or steps not expressly listed or inherent to such setup or device or method. In other words, one or more elements in a device or system or apparatus proceeded by “comprises . . . a” does not, without more constraints, preclude the existence of other elements or additional elements in the device or system or apparatus.

In the following detailed description of the embodiments of the disclosure, reference is made to the accompanying drawings that form a part hereof, and in which are shown by way of illustration specific embodiments in which the disclosure may be practiced. These embodiments are described in sufficient detail to enable those skilled in the art to practice the disclosure, and it is to be understood that other embodiments may be utilized and that changes may be made without departing from the scope of the present disclosure. The following description is, therefore, not to be taken in a limiting sense.

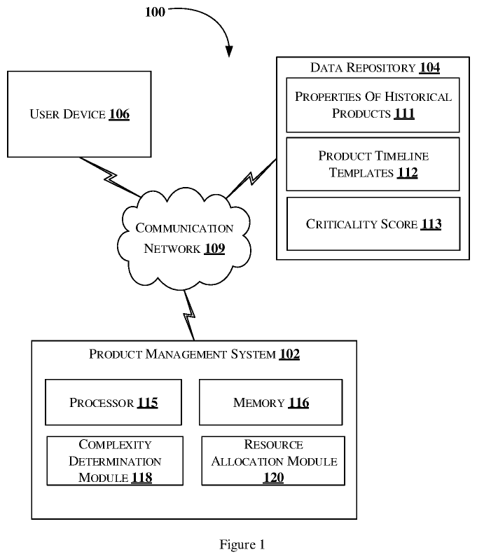

FIG. 1 illustrates an exemplary architecture of a proposed system to allocate resources for product development based on complexity of the product in accordance with some embodiments of the present disclosure.

As illustrated in FIG. 1, the exemplary system 100 comprises one or more components configured for effectively assigning resources for product development. In one embodiment, the exemplary system 100 comprises a product management system (hereinafter referred to as PMS) 102, a data repository (interchangeably referred to as repository) 104, and a user device 106 connected via a communication network 109. The communication network 109 can be a LAN (local area network), WAN (wide area network), wireless network, point-to-point network, or another configuration. One of the most common types of network in current use is a TCP/IP (Transfer Control Protocol and Internet Protocol) network for communication between database client and database server. Other common Internet protocols used for such communication include HTTPS, FTP, AFS, and WAP and using secure communication protocols etc.

The data repository 104 stores properties associated with each category of historical products 111. In one embodiment one or more categories can be Active pharmaceutical ingredient (API), dosage form, manufacturing process, formulation composition, stability, analytical testing, and reference product. In one example, the properties associated with each category of the historical products 111 may be a historical data collected from plurality of products developed previously. The properties associated with each category of the historical products 111, in one example for a pharmaceutical product or molecule, may comprise properties such as flow properties, hardness, friability, weight variation, stickiness, and other related properties of the molecule or the pharmaceutical product. The data repository 104 also stores classification associated with the historical products 111 along with the properties. The data repository 104 further stores a predefined plurality of project timeline templates 112 associated with each classification of product. The plurality of project timeline templates 112 may be predetermined using resources assigned for the previously developed products. Further, the data repository 104 stores information about each historical product, for example molecule details, bio-study synopsis and bio-study protocol. The data repository 104 also stores criticality score 113 associated with each category for historical products developed previously. For example, the criticality score 113 associated with each category may be used to compute complexity for developing the historical products and is determined using number of resources involved, number of test procedures associated and so on. In one embodiment, the data repository 104 may be integrated within the PMS 102. In another embodiment, the data repository 104 may be a standalone repository communicatively coupled with the PMS 102.

The user device 106 may be a mobile device or a computing device including the functionality for communicating over the network 109. For example, the mobile device can be a conventional web-enabled personal computer in the home, mobile computer (laptop, notebook or subnotebook), Smart Phone (iOS, Android), personal digital assistant, wireless electronic mail device, tablet computer or other device capable of communicating both ways over the Internet or other appropriate communications network. The user device 106 may comprise an integrated software application with a user interface that enables interaction with the PMS 102. The integrated software application receives input from the user such as product name, one or more categories of the product, developer names etc. and enables users to view estimated timeline and resource allocation data determined by the PMS 102. In one example, the user device 106 may be communicatively coupled with the PMS 102.

The PMS 102 is configured to assign resources for the product development effectively by determining complexity score of development of the product. In one example, the PMS 102 may be configured as a standalone system. In another example, the PMS 102 may be configured in cloud environment. In yet another example, the PMS 102 may include a desktop personal computer, workstation, laptop, PDA, cell phone, or any WAP-enabled device or any other computing device capable of interfacing directly or indirectly to the Internet or other network connection. The PMS 102 typically includes one or more user interface devices, such as a keyboard, a mouse, touch screen, pen or the like, for interacting with the GUI provided on a display. The PMS 102 also includes a graphical user interface (GUI) provided therein for interacting with the data repository 104 to access the plurality of properties of historical products 111, the project timeline templates 112 and the criticality score 113.

The PMS 102 comprises at least a processor 115 and a memory 116 coupled with the processor 115. The PMS 102 further comprises a complexity determination module 118 and a resource allocation module 120. The complexity determination module 118 is configured to determine complexity score associated with development of the product based on properties of the product to be developed. The resource allocation module 120 is configured to determine resources i.e., developers or project members, equipment required for development of the product and assign resources with appropriate tasks of development of the product.

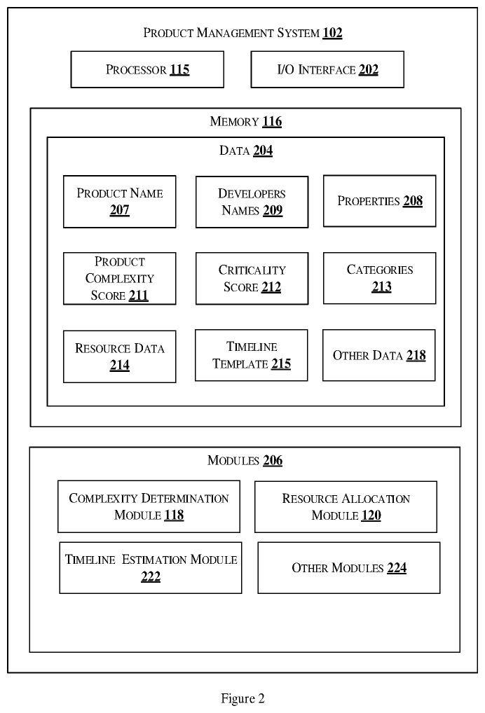

In an embodiment, the PMS 102 may be a typical PMS as illustrated in FIG. 2. The PMS 102 comprises the processor 115, the memory 116, and an I/O interface 202. The I/O interface 202 is coupled with the processor 115 and an I/O device (not shown). The I/O device is configured to allow the processor 115 to communicate with and control from various I/O devices. The I/O device is configured to receive inputs via the I/O interface 202 and transmit outputs via the I/O interface 202. The PMS 102 further includes data 204 and one or more modules 206. In one implementation, data 204 may be stored within the memory 116. In one example, the data 204 may include product name 207, properties 208, developers names 209, product complexity score 211, criticality score 212 associated with each category of the product, one or more categories 213 of the product, resource data 214, timeline template 215, and other data 218. In some embodiments, the data 204 may be stored within the memory 116 in the form of various data structures. Additionally, the data 204 may be organized using data models, such as relational or hierarchical data models. The other data 218 may store temporary data and temporary files, generated by the components for performing the various functions of the PMS 102.

The modules 206 may include, for example the complexity determination module 118, the resource allocation module 120, and a timeline estimation module 222. The complexity determination module 118 is configured to determine criticality score of each category of the product based on comparison of properties associated with each category of the current product to be developed with the properties of the historical products 111. The complexity determination module 118 is further configured to determine the product complexity score 211 of the current product based on criticality score 212 of all categories of the product and also a weightage value for each category of the product. Upon determining the product complexity score 211, the timeline estimation module 222 classifies the product based on the product complexity score 211. For example, the timeline estimation module 222 classifies the product into one or more classification 213 such as for example, simple Immediate Release (IR), complex Immediate Release (IR), simple Extended Release (ER), complex Extended Release (ER) and so on. Based on the classification 213 of the product, the timeline estimation module 222 retrieves the predefined project timeline template 112 from the repository 104.

The resource allocation module 120 is configured to determine the resource data 214 comprising availability of resources, equipment required etc., to complete the product development process. Using the resource data 214 determined by the resource allocation module 120, the timeline estimation module 222 updates the project timeline template 112 and determines a timeline for development of the product.

The modules 206 may comprise other modules 224 to perform various miscellaneous functionality of the PMS 102. It will be appreciated that such aforementioned modules may be represented as a single module or a combination of different modules. The modules may be implemented in the form of software executed by a processor, hardware and/or firmware.

As an initial step, the PMS 102 receives the product name 207, one or more categories 213 of the product, one or more properties 208 associated with each category of the product, and developers names 209, and determines the complexity score 211 of development of the product. In one embodiment, the processor 115 is configured to compare the product name 207 with name of the one or more historical products and determine count of the one or more categories of the product based on the comparison, so as to determine the one or more categories required to be input from the user. In another embodiment, one or more categories of the product are automatically determined when the product name 207 matches with name of one or more historical products and displayed to the user for receiving confirmation from the user, thereby eliminating a need for the user to manually decide on one or more categories required if the product to be developed is same as one of the historical products. The user or authorized person may input the product name 207 i.e., name of the product that is to be developed, one or more categories 213 of the product, one or more properties 208 associated with each category the product and developers names 209 i.e., names of one or more developers or project members who participate in development of the product via the integrated software application of the user device 106. In one embodiment, the user may input data via the I/O interface 202. For example, the product may be a pharmaceutical product or molecule and the category of the product may include formulation composition, stability, reference product, dosage form and so on. In one embodiment, the complexity determination module 118 receives the product name 207, developer's names 209, categories 213, and properties 208 associated with each category of the product and searches for the same or similar product in the repository 104. The complexity determination module 118 compares the properties 208 associated with each category of the product with the plurality of properties of the historical products 111 stored in the repository 104 to determine similarity of the product with respect to the historical products 111. If the properties 208 associated with each category of the product matches with the properties of the historical products 111, the complexity determination module 118 determines the product complexity score 211 for development of the product using criticality score 113 for the same or similar historical products developed previously and a weightage value corresponding to each category. If the properties 208 associated with each category of the product do not match with the properties of the historical products 111, the complexity determination module 118 classifies each of the properties of the historical products 111 present in the repository 104 as one of continuous property or discrete property.

The continuous property is defined as the property of a product that is assigned with any value between a preferred range of values. The discrete property is defined as the property of a product that is assigned with countable number of values. in a range. Upon classifying the properties of the historical products 111 into one of continuous and discrete properties, the complexity determination module 118 normalizes values of one of the classified continuous and discrete properties to improve computational efficiency and minimize database complexity. The complexity determination module 118 normalizes each of the continuous properties into a range of 0 and 1. Further, the complexity determination module 118 normalizes each of the discrete properties using known normalization techniques. Upon normalizing the properties of the historical products 111, the complexity determination module 118 compares each of the properties 208 associated with each category of the product with the corresponding normalized property of the historical products 111 and determines the similar historical product having properties matching to each property 208 associated with each category of the product. The complexity determination module 118 further determines the criticality score 212 for each category of the product using the predefined criticality score 113 of the corresponding similar historical product with respect to the corresponding property. Upon determining the criticality score 212 of each category of the product, the complexity determination module 118 determines the product complexity score 211 by using the criticality score 212 of each category of the product and a weightage value corresponding to each category. In one embodiment, the weightage value corresponding to each category of the product is dynamically updated based on comparison of the product complexity score 211 with an actual complexity score, wherein the actual complexity score is received from the user after development of the product.

Upon determining the product complexity score 211, the timeline estimation module 222 classifies the product based on the product complexity score 211. For example, the timeline estimation module 222 classifies the product into one or more classification 213 such as for example, simple Immediate Release (IR), complex Immediate Release (IR), simple Extended Release (ER), complex Extended Release (ER) and so on. Based on the classification 213 of the product, the timeline estimation module 222 retrieves the predefined project timeline template 112 from the repository 104. The project timeline template 112 may comprise number of man days and machine days required to complete one or more tasks of development of the product. The resource allocation module 120 determines the resource data 214 such as availability of resources i.e., developers or other project members required to accomplish multiple tasks for development of the product, equipment required and current workload of the developers or other project members, using the developer names and one or more internal portals associated with an organization under which the product is developed.

Using the resource data 214 determined by the resource allocation module 120, the timeline estimation module 222 updates the project timeline template 112 and determines a timeline for development of the product.

Thus, using the embodiments implemented, the system achieves efficient resource assignment by accurate determination of complexity of the project before initiating the project development process. The system also dynamically generates the complexity score for a new product development based on minimal inputs without requiring to conduct any experiments, thereby improving accuracy in the complexity evaluation to a greater extent. Further, the system also generates a more accurate timeline and resource estimation for the new product development with minimal inputs from the user.

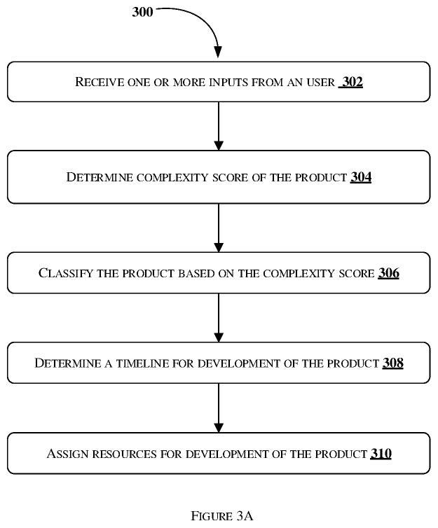

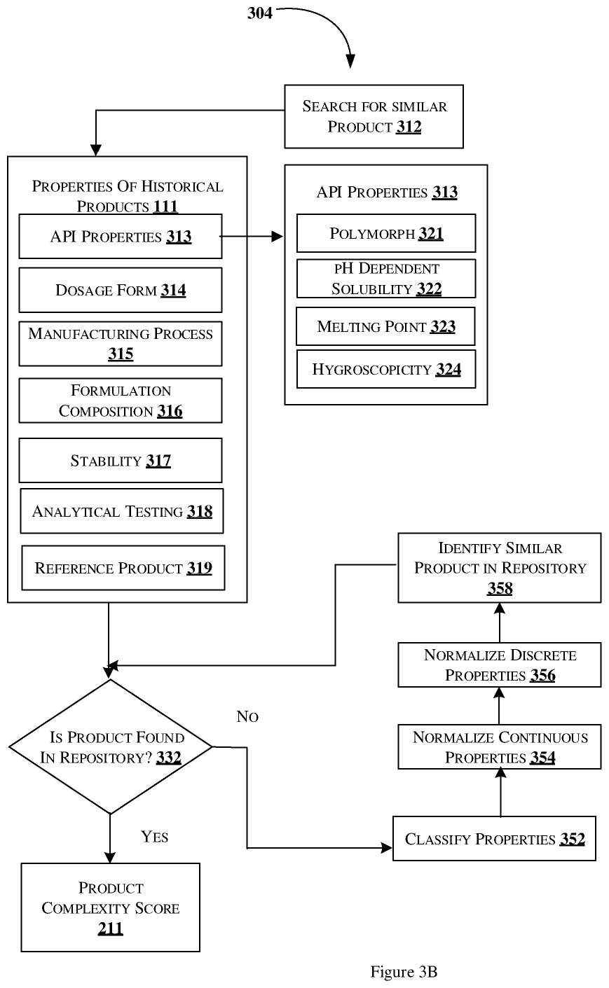

FIG. 3A illustrates an exemplary steps of project management and resource allocation in accordance with an embodiment of the present disclosure.

As illustrated in FIG. 3a, the method 300 comprises one or more blocks implemented by the processor 115 for effectively assigning resources based on complexity of product development. The method 300 may be described in the general context of computer executable instructions. Generally, computer executable instructions can include routines, programs, objects, components, data structures, procedures, modules, and functions, which perform particular functions or implement particular abstract data types.

The order in which the method 300 is described is not intended to be construed as a limitation, and any number of the described method blocks can be combined in any order to implement the method 300. Additionally, individual blocks may be deleted from the method 300 without departing from the spirit and scope of the subject matter described herein. Furthermore, the method 300 can be implemented in any suitable hardware, software, firmware, or combination thereof.

At block 302, one or more inputs from an user are received. In one embodiment, the one or more inputs include product name 207, one or more categories 213, one or more properties 208 associated with each category of the product and developers names. The user or authorized person inputs product name 207 i.e., name of the product that is to be developed, properties 208 associated with each category of the product and developers names 209 i.e., names of one or more developers or project members who participate in development of the product via the integrated software application of the user device. For example, the product may be a pharmaceutical product or molecule. In one embodiment, the complexity determination module 118 receives the product name 207, developer's names 209, categories 213, and properties 208 associated with each category of the product. In one embodiment, properties associated with one or more categories of the product are automatically determined when the product name 207 matches with name of one or more historical products and displayed to the user for receiving confirmation from the user, thereby eliminating a need for the user to input one or more categories if the product to be developed is same as one of the historical products. In another embodiment, the processor is configured to compare product name 207 with the name of one or more historical products and determine count of the one or more categories of the product based on the comparison.

At block 304, complexity score of the product is determined as depicted in FIG. 3b. The complexity determination module 118 is configured to determine the product complexity score 211 of the current product based on criticality score 212 of all categories of the product and also a weightage value for each category of the product.

At block 306, the timeline estimation module 222 classifies the product based on the product complexity score 211. For example, the classification 213 of a pharmaceutical product may include one of simple Immediate Release (IR), complex Immediate Release (IR), simple Extended Release (ER), complex Extended Release (ER) and so on.

At block 308, Based on the classification 213 of the product, the timeline estimation module 222 retrieves the predefined project timeline template 112 from the repository 104. The project timeline template 112 may comprise number of man days and machine days required to complete one or more tasks of development of the product. The resource allocation module 120, after retrieving the predefined project timeline template, determines the resource data 214 such as availability of resources i.e., developers or other project members required to accomplish multiple tasks for development of the product, equipment required and current workload of the developers or other project members, using the developer names and one or more internal portals associated with an organization under which the product is developed. Using the resource data 214 determined by the resource allocation module 120, the timeline estimation module 222 updates the project timeline template 112 determines a timeline required for development of the product.

At block 310, the resource allocation module 120 assign the resources for one or more tasks of development of the product upon confirmation from the user and updating the data repository 104 with the assigned resources upon receiving confirmation from the user.

Thus, using the embodiments implemented, the system achieves efficient resource allocation by accurate determination of complexity of the project before initiating the project development process.

FIG. 3B illustrates an exemplary steps of complexity evaluation of product development in accordance with an embodiment of the present disclosure.

As illustrated in FIG. 3B, the method 304 comprises one or more blocks implemented by the processor 115 for determining the complexity score of the product. The method 304 may be described in the general context of computer executable instructions. Generally, computer executable instructions can include routines, programs, objects, components, data structure, procedures, modules, and functions, which perform particular functions or implement particular abstract data types.

The order in which the method 304 is described is not intended to be construed as a limitation, and any number of the described method blocks can be combined in any order to implement the method 304. Additionally, individual blocks may be deleted from the method 350 without departing from the spirit and scope of the subject matter described herein. Furthermore, the method 304 can be implemented in any suitable hardware, software, firmware, or combination thereof.

At block 312, a search is made to determine same or similar product in the repository 114. In one embodiment, the complexity determination module 118 compares the properties 208 associated with each category of the product with the plurality of properties of the historical products 111 stored in the repository 104 to determine similarity of the product with respect to the historical products or previously developed products. For example, for a pharmaceutical product, the categories of historical products 111 may comprise Active pharmaceutical ingredient (API) properties 313, dosage form 314, manufacturing process 315, formulation composition 316, stability 317, analytical testing 318 and reference product 319. Each category 213 of the historical products 111 comprises one or more properties. For example, the API properties 313 may comprise one or more properties such as polymorph 321, pH dependent solubility 322, melting point 323 and hygroscopicity 324.

The complexity determination module 118 determines whether the same or similar product is available in the repository 104. If the properties 208 associated with each category of the product matches with the properties of the historical products 111 (YES path), the complexity determination module 118 determines the product complexity score 211 for development of the product using the criticality score 113 for the same or similar historical product developed previously. If the properties 208 of the product do not match with the properties of the historical products 111 (NO path), the complexity determination module 118 classifies each of the properties of the historical products 111 present in the repository 104 as one of continuous property or discrete property (at block 352). The continuous property is defined as the property of a product that is assigned with any value between a preferred range of values. The discrete property is defined as the property of a product that is assigned with countable number of values in a range between any two values. Upon classifying the properties of historical products 111 into one of continuous and discrete properties, the complexity determination module 118 normalizes values of one of the classified continuous and discrete properties to improve computational efficiency and minimize database complexity. The complexity determination module 118 normalizes each of the continuous properties into a range of 0 and 1 (at block 354).

Further, the complexity determination module 118 normalizes each of the discrete properties using known normalization techniques(at block 356). Upon normalizing the properties of the historical products 111, the complexity determination module 118 compares each of the properties 208 associated with each category of the product with the corresponding normalized property of the historical products 111 and determines the similar historical product having properties matching to each property 208 of the product (at block 358). The complexity determination module 118 determines the criticality score 212 for each category of the product using the predefined criticality score 113 of the corresponding similar historical product with respect to the corresponding property. Upon determining the complexity score 212 of each category of the product, the complexity determination module 118 determines the product complexity score 211 by using the criticality score 212 of each category of the product and a weightage value corresponding to each category. In one embodiment, the weightage value corresponding to each category of the product is dynamically updated based on comparison of the product complexity score 211 with an actual complexity score, wherein the actual complexity score is received from the user an input after development of the product.

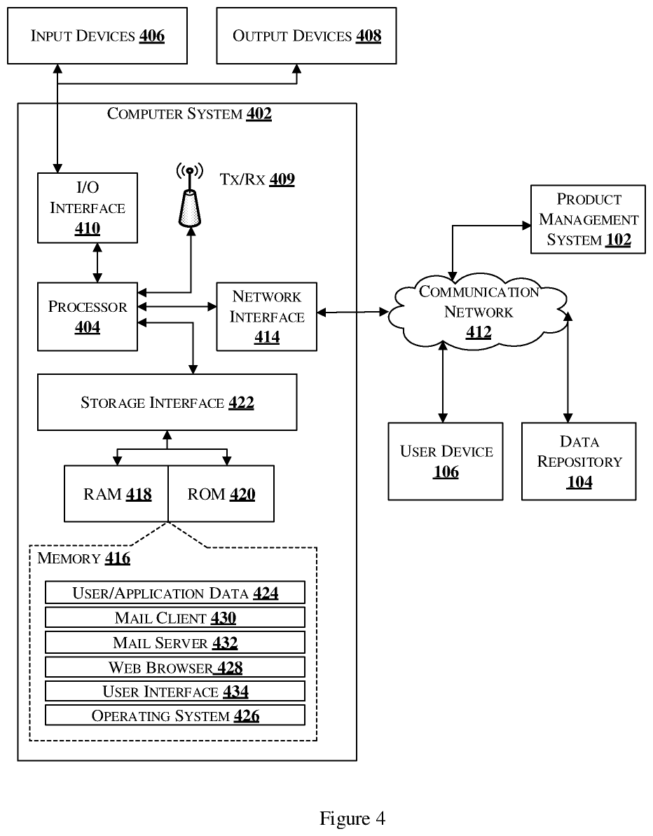

FIG. 4 illustrates a block diagram of an exemplary computer system for implementing embodiments consistent with the present disclosure.

In an embodiment, the computer system 402 may be product management system 102, which is used for effective allocation of resources based on product complexity. The computer system 402 may include a central processing unit (“CPU” or “processor”) 404. The processor 404 may comprise at least one data processor for executing program components for executing user or system-generated business processes. The processor 404 may include specialized processing units such as integrated system (bus) controllers, memory management control units, floating point units, graphics processing units, digital signal processing units, etc.

The processor 404 may be disposed in communication with one or more input/output (I/O) devices (406 and 408) via I/O interface 410.The I/O interface 410 may employ communication protocols/methods such as, without limitation, audio, analog, digital, stereo, IEEE-1394, serial bus, Universal Serial Bus (USB), infrared, PS/2, BNC, coaxial, component, composite, Digital Visual Interface (DVI), high-definition multimedia interface (HDMI), Radio Frequency (RF) antennas, S-Video, Video Graphics Array (VGA), IEEE 802.n/b/g/n/x, Bluetooth, cellular (e.g., Code-Division Multiple Access (CDMA), High-Speed Packet Access (HSPA+), Global System For Mobile Communications (GSM), Long-Term Evolution (LTE) or the like), etc.

Using the I/O interface 410, the computer system 402 may communicate with one or more I/O devices. For example, the input device 406 may be an antenna, keyboard, mouse, joystick, (infrared) remote control, camera, card reader, fax machine, dongle, biometric reader, microphone, touch screen, touchpad, trackball, sensor (e.g., accelerometer, light sensor, GPS, gyroscope, proximity sensor, or the like), stylus, scanner, storage device, transceiver, video device/source, visors, etc. Output device 408 may be a printer, fax machine, video display (e.g., cathode ray tube (CRT), liquid crystal display (LCD), light-emitting diode (LED), plasma, or the like), audio speaker, etc. In some embodiments, a transceiver 409 may be disposed in connection with the processor 404. The transceiver may facilitate various types of wireless transmission or reception. For example, the transceiver may include an antenna operatively connected to a transceiver chip (e.g., Texas Instruments WiLink WL1283, Broadcom BCM4750IUB8, Infineon Technologies X-Gold 618-PMB9800, or the like), providing IEEE 802.11a/b/g/n, Bluetooth, FM, global positioning system (GPS), 2G/3G HSDPA/HSUPA communications, etc.

In some implementations, the processor 404 may be disposed in communication with a communication network 412 via a network interface 414. The network interface 414 may employ connection protocols including, without limitation, direct connect, Ethernet (e.g., twisted pair 10/100/1000 Base T), Transmission Control Protocol/Internet Protocol (TCP/IP), token ring, IEEE 802.11a/b/g/n/x, etc. Using the network interface 414 and the communication network 412, the computer system 402 may be connected to the product management system102, user device 106 and the data repository 104.

The communication network 412 can be implemented as one of the several types of networks, such as intranet or any such wireless network interfaces. The communication network 412 may either be a dedicated network or a shared network, which represents an association of several types of networks that use a variety of protocols, for example, Hypertext Transfer Protocol (HTTP), Transmission Control Protocol/Internet Protocol (TCP/IP), Wireless Application Protocol (WAP), etc., to communicate with each other. Further, the communication network 412 may include a variety of network devices, including routers, bridges, servers, computing devices, storage devices, etc.

In some embodiments, the processor 404 may be disposed in communication with a memory 416 e.g., RAM 418, and ROM 420, etc. as shown in FIG. 4, via a storage interface 422. The storage interface 422 may connect to memory 416 including, without limitation, memory drives, removable disc drives, etc., employing connection protocols such as Serial Advanced Technology Attachment (SATA), Integrated Drive Electronics (IDE), IEEE-1394, Universal Serial Bus (USB), fiber channel, Small Computer Systems Interface (SCSI), etc. The memory drives may further include a drum, magnetic disc drive, magneto-optical drive, optical drive, Redundant Array of Independent Discs (RAID), solid-state memory devices, solid-state drives, etc.

The memory 416 may store a collection of program or database components, including, without limitation, user/application 424, an operating system 426, a web browser 428, a mail client 430, a mail server 432, a user interface 434, and the like. In some embodiments, computer system 402 may store user/application data 424, such as the data, variables, records, etc. as described in this invention. Such databases may be implemented as fault-tolerant, relational, scalable, secure databases such as Oracle or Sybase.

The operating system 426 may facilitate resource management and operation of the computer system 402. Examples of operating systems include, without limitation, Apple Macintosh™ OS X™, UNIX™, Unix-like system distributions (e.g., Berkeley Software Distribution (BSD), FreeBSD™, Net BSD™, Open BSD™, etc.), Linux distributions (e.g., Red Hat™, Ubuntu™, K-Ubuntu™, etc.), International Business Machines (IBM™) OS/2™, Microsoft Windows™ (XP™, Vista/7/8, etc.), Apple iOS™, Google Android™, Blackberry™ Operating System (OS), or the like. A user interface may facilitate display, execution, interaction, manipulation, or operation of program components through textual or graphical facilities. For example, user interfaces may provide computer interaction interface elements on a display system operatively connected to the computer system 402, such as cursors, icons, check boxes, menus, windows, widgets, etc. Graphical User Interfaces (GUIs) may be employed, including, without limitation, Apple™ Macintosh™ operating systems' Aqua™, IBM™ OS/2™, Microsoft™ Windows™ (e.g., Aero, Metro, etc.), Unix X-Windows™, web interface libraries (e.g., ActiveX, Java, JavaScript, AJAX, HTML, Adobe Flash, etc.), or the like.

The illustrated steps are set out to explain the exemplary embodiments shown, and it should be anticipated that ongoing technological development will change the manner in which particular functions are performed. These examples are presented herein for purposes of illustration, and not limitation. Further, the boundaries of the functional building blocks have been arbitrarily defined herein for the convenience of the description. Alternative boundaries can be defined so long as the specified functions and relationships thereof are appropriately performed. Alternatives (including equivalents, extensions, variations, deviations, etc., of those described herein) will be apparent to persons skilled in the relevant art(s) based on the teachings contained herein. Such alternatives fall within the scope and spirit of the disclosed embodiments. Also, the words “comprising,” “having,” “containing,” and “including,” and other similar forms are intended to be equivalent in meaning and be open ended in that an item or items following any one of these words is not meant to be an exhaustive listing of such item or items, or meant to be limited to only the listed item or items. It must also be noted that as used herein and in the appended claims, the singular forms “a,” “an,” and “the” include plural references unless the context clearly dictates otherwise.

Furthermore, one or more computer-readable storage media may be utilized in implementing embodiments consistent with the present disclosure. A computer-readable storage medium refers to any type of physical memory on which information or data readable by a processor may be stored. Thus, a computer-readable storage medium may store instructions for execution by one or more processors, including instructions for causing the processor(s) to perform steps or stages consistent with the embodiments described herein. The term “computer-readable medium” should be understood to include tangible items and exclude carrier waves and transient signals, i.e., are non-transitory. Examples include random access memory (RAM), read-only memory (ROM), volatile memory, non-volatile memory, hard drives, CD ROMs, DVDs, flash drives, disks, and any other known physical storage media.

Finally, the language used in the specification has been principally selected for readability and instructional purposes, and it may not have been selected to delineate or circumscribe the inventive subject matter. Accordingly, the disclosure of the embodiments of the disclosure is intended to be illustrative, but not limiting, of the scope of the disclosure.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

1. A method of assigning resources for development of a pharmaceutical product, comprising:
receiving one or more inputs associated with the pharmaceutical product from a user;
determining a complexity score of the pharmaceutical product based on the one or more inputs received;
classifying the pharmaceutical product based on the complexity score;
determining a timeline for development of the pharmaceutical product based on the classification; and
assigning the resources for development of the pharmaceutical product based on the determination, wherein the resources include at least one of project members and equipment's required for development of the pharmaceutical product.

2. The method as claimed in claim 1, wherein the one or more inputs includes name of the pharmaceutical product, one or more categories of the pharmaceutical product, and name of the user, and wherein the one or more categories includes at least one of Active pharmaceutical ingredient (API), dosage form, manufacturing process, formulation composition, stability, analytical testing, and reference product.

3. The method as claimed in claim 2, wherein determining the complexity score of the pharmaceutical product includes:
comparing one or more properties of each category with a plurality of properties of one or more historical pharmaceutical products stored in a data repository;
assigning a criticality score for each category based on the comparison; and
determining the complexity score of the pharmaceutical product based on the criticality score of each category, and a weightage value corresponding to each category.

4. The method as claimed in claim 3, wherein assigning the criticality score for each category comprises:
assigning a criticality score for each category when the one or more properties of the pharmaceutical products best matches with the plurality of properties of the one or more historical pharmaceutical products.

5. The method as claimed in claim 3, wherein assigning the criticality score for each category comprises:
classifying the plurality of properties of the one or more historical pharmaceutical products to one of continuous and discrete properties if the one or more properties of the pharmaceutical products do not match with the plurality of properties of the one or more historical pharmaceutical products;
normalizing values of the continuous properties into corresponding values of the discrete properties; and
assigning a criticality score of the normalized values of discrete properties to each category.

6. The method as claimed in claim 3, further comprising:
receiving an actual complexity score from the user as input after development of the pharmaceutical product; and
dynamically adjusting the weightage value corresponding to each category based on comparison of the complexity score with the actual complexity score received.

7. The method as claimed in claim 1, wherein determining the timeline for development of the pharmaceutical product includes:
identifying a timeline template for the pharmaceutical product based on the classification, wherein the classification includes one of simple Immediate Release (IR), complex Immediate Release (IR), simple Extended Release (ER), and complex Extended Release (ER), wherein the timeline template comprises a number of project member days and equipment days required for development of the pharmaceutical product;
determining the resources required based on the complexity score and current workload of the resources;
updating the timeline template identified based on the determination; and
determining the timeline for development of the pharmaceutical product based on the timeline template updated.

8. The method as claimed in claim 1, further comprising updating the data repository with the assigned resources upon receiving confirmation from the user.

9. The method as claimed in claim 2, further comprising:
comparing the name of the pharmaceutical product with name of the one or more historical pharmaceutical products; and
determining count of the one or more categories of the pharmaceutical product based on the comparison, so as to input the one or more categories required by the user.

10. The method as claimed in claim 2, wherein the one or more categories of the pharmaceutical product are automatically determined if the name of the pharmaceutical product matches with name of one or more historical pharmaceutical products and displayed to the user for receiving confirmation from the user.

11. A system to assign resources for development of a pharmaceutical product, comprising:
a memory; and
a processor, coupled to the memory, and is configured to:
receive one or more inputs associated with the pharmaceutical product from a user;
determine a complexity score of the pharmaceutical product based on one or more inputs received;
classify the pharmaceutical product based on the complexity score;
determine a timeline for development of the pharmaceutical product based on the classification; and
assign the resources for development of the pharmaceutical product based on the determination, wherein the resources include at least one of project members and equipment's required for development of the pharmaceutical product.

12. The system as claimed in claim 11, wherein the one or more inputs includes name of the pharmaceutical product, one or more categories of the pharmaceutical product, and name of the user, and wherein the one or more categories includes at least one of Active pharmaceutical ingredient (API), dosage form, manufacturing process, formulation composition, stability, analytical testing, and reference product.

13. The system as claimed in claim 12, wherein to determine the complexity score of the pharmaceutical product, the processor is configured to:
compare the one or more properties of each category with a plurality of properties of one or more historical pharmaceutical products stored in a data repository;
assign a criticality score for each category based on the comparison; and
determine the complexity score of the pharmaceutical product based on the criticality score of each category, and a weightage value corresponding to each category.

14. The system as claimed in claim 13, wherein the processor is configured to assign the criticality score for each category by:
assigning a criticality score for each category when the one or more properties of the pharmaceutical products best matches with the plurality of properties of the one or more historical pharmaceutical products.

15. The system as claimed in claim 13, wherein the processor is configured to assign the criticality score for each category by:
classifying the plurality of properties of the one or more historical pharmaceutical products to one of continuous and discrete properties when the one or more properties of the pharmaceutical products do not match with the plurality of properties of the one or more historical pharmaceutical products;
normalizing values of the continuous properties into corresponding values of the discrete properties; and
assigning a criticality score of the normalized values of discrete properties to each category.

16. The system as claimed in claim 13, wherein the processor is configured to:
receive an actual complexity score from the user as input after development of the pharmaceutical product; and
dynamically adjust the weightage value corresponding to each category based on comparison of the complexity score with the actual complexity score received.

17. The system as claimed in claim 11, wherein to determine the timeline for development of the pharmaceutical product, the processor is configured to:
identify a timeline template for the pharmaceutical product based on the classification, wherein the classification includes one of simple Immediate Release (IR), complex Immediate Release (IR), simple Extended Release (ER), and complex Extended Release (ER), wherein the timeline template comprises a number of project member days and equipment days required for development of the pharmaceutical product;
determine the resources required based on the complexity score and current workload of the resources;
update the timeline template identified based on the determination; and
determine the timeline for development of the pharmaceutical product based on the timeline template updated.

18. The system as claimed in claim 11, wherein the processor is configured to update the date repository with the assigned resources upon receiving confirmation from the user.

19. The system as claimed in claim 12, wherein processor is configured to:
compare the name of the pharmaceutical product with name of the one or more historical pharmaceutical products; and
determine count of the one or more categories of the pharmaceutical product based on the comparison, so as to input the one or more categories required by the user.

20. The system as claimed in claim 12, wherein the processor is configured to:
automatically determine the one or more categories of the pharmaceutical products if the name of the pharmaceutical product matches with name of one or more historical pharmaceutical products; and
display the determined one or more categories for receiving confirmation from the user.
